(19) United States
(12) Patent Application Publication
(10) Pub. No.: US 2016/0256466 A1
(43) Pub. Date: Sep. 8, 2016

(54) PI3K INHIBITOR FOR TREATMENT OF RESPIRATORY DISEASE

(71) Applicant: GLAXOSMITHKLINE INTELLECTUAL PROPERTY DEVELOPMENT LIMITED, Brentford Middlesex (GB)

(72) Inventors: Augustin AMOUR, Stevenage, Hertfordshire (GB); Julie Nicole HAMBLIN, Stevenage, Hertfordshire (GB); Edith HESSEL, Stevenage, Hertfordshire (GB); David MICHALOVICH, Stevenage, Hertfordshire (GB); Srividya SRISKANTHARAJAH, Stevenage, Hertfordshire (GB)

(73) Assignee: GlaxoSmithKline Intellectual Property Development Limited, Brentford, Middlesex (GB)

(21) Appl. No.: 15/028,954

(22) PCT Filed: Oct. 15, 2014
(86) PCT No.: PCT/EP2014/072073

§ 371 Date: Apr. 13, 2016

(30) Foreign Application Priority Data
Oct. 17, 2013 (GB) 1318414.8
Nov. 11, 2013 (GB) 1319824.7
May 21, 2014 (GB) 1409014.6

(51) Int. Cl. A61K 31/5377; C12Q 1/48; A61K 31/496

(52) U.S. Cl. CPC A61K 31/5377; A61K 31/496; C12Q 1/485; G01N 2800/52; G01N 2333/91215

(57) ABSTRACT

The present invention is directed to compounds and pharmaceutically acceptable salts thereof which are inhibitors of the activity or function of the phosphoinositide 3′OH kinase family (hereinafter PI3K) for use in the treatment or prevention of respiratory infections, the treatment of airway damage, and/or the prevention of airway injury in patients with a PI3Kδ mutation.

### FIELD OF THE INVENTION

The present invention is directed to compounds and pharmaceutically acceptable salts thereof which are inhibitors of the activity or function of the phosphoinositide 3′OH kinase family (hereinafter PI3K), in particular PI3Kδ, for use in the treatment or prevention of respiratory infections, the treatment of airway damage, and/or the prevention of airway injury in patients with a PI3Kδ mutation.

### BACKGROUND OF THE INVENTION

The Class I PI3 kinase family comprises 4 separate isoforms (α, β, γ and δ) distinguished by the sequence and structure of the p110 catalytic subunit. A number of different genetic variants in PI3Kδ have been observed (Jou et al., International Journal of Immunogenetics, 2006, 33, 361 to 369; Angulo et al., Science DOI: 10.1126/science. 1243292; Lucas et al., Nature Immunology DOI: 10.1038/ni.2271; Crank et al., J. Clin. Immunol., DOI 10.1007/s 10875-014-0012-9; and Deau et al., J. Clin. Invest., DOI:10.1172/JCI75746). Some genetic variants may result in silent nucleotide exchange which does not lead to amino acid substitution whereas others result in amino acid substitution in regions outside the catalytic centre, for example an asparagine to serine substitution at codon 253 in the Ras-binding domain and an alanine to threonine substitution in exon 11. Other mutations include a mutation (m.3256G>A) observed in a highly conserved position in the domain responsible for catalytic function that resulted in a glutamic acid to lysine substitution (E1021K); a C to A mutation at cDNA position 1002 that resulted in the amino acid substitution N334K in the C2 domain; a G to A mutation at nucleotide 1573 that resulted in a E525K substitution in the helical domain; a gain of function mutation of PIK3CD c. 1246T>C, p. C416R; and a PIK3R1 mRNA splice mutation resulting in the exclusion of exon 10 and thus the deletion of amino acid residues 434-475 of the p85α regulatory subunits.

Although the mechanism of activation of PI3Kδ mutation is not understood at a molecular level, PI3K is activated by interaction with other protein targets and also by domain-domain interactions within the protein itself. Mutations that change the function of PI3Kδ may therefore arise both inside and outside the catalytic functional domain. Such mutations may lead to a change in the stability of the folded protein, a change in expression levels and/or a change in interactions with other proteins. Thus mutations in PI3Kδ may lead to inappropriate PI3Kδ activity, which may be increased or decreased as compared to wild type protein.

The role of PI3Kδ mutations in immunodeficiency has been reported (Jou et al., International Journal of Immunogenetics, 2006, 33, 361 to 369, Angulo et al., Science DOI: 10.1126/science. 1243292, and Lucas et al., Nature Immunology DOI: 10.1038/ni.2271). Patients with PI3Kδ mutations may be particularly susceptible to developing respiratory infections and/or exacerbations of respiratory infections, and damage to the airway wall, large and small airways, and lung parenchyma. There thus remains a need to provide novel therapeutics for patients with a PI3Kδ mutation.

The present invention provides compounds and pharmaceutically acceptable salts thereof which are inhibitors of the activity or function of PI3Kδ for use in the treatment or prevention of respiratory infections, the treatment of airway damage, and/or the prevention of airway injury in patients with a PI3Kδ mutation.

### SUMMARY OF THE INVENTION

The present invention provides compounds of formula (I)

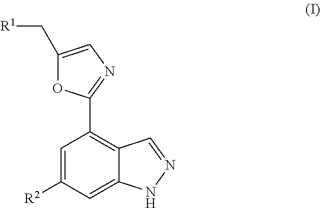

wherein R¹ and R² are as defined below, and pharmaceutically acceptable salts thereof, for use in the treatment or prevention of respiratory infections, the treatment of airway damage, and/or the prevention of airway injury in patients with a PI3Kδ mutation.

In one embodiment, the present invention provides compounds of formula (I) and pharmaceutically acceptable salts thereof for use in the treatment or prevention of respiratory infections, the treatment of airway damage, and/or the prevention of airway injury in patients comprising assaying samples from the patients, determining if the patients have a PI3Kδ mutation, and administering a therapeutically effective amount of a compound of formula (I) or a pharmaceutically acceptable salt thereof to the patients if they have a PI3Kδ mutation.

In another embodiment, the invention provides compounds of formula (I) and pharmaceutically acceptable salts thereof for use in the treatment or prevention of respiratory infections, the treatment of airway damage, and/or the prevention of airway injury in patients classified as responders, wherein a responder is characterised by the presence of a PI3Kδ mutation.

In a further embodiment, the invention provides a method of evaluating therapy with compounds of formula (I) and pharmaceutically acceptable salts thereof, comprising obtaining a sample from the patient, testing for a PI3Kδ mutation, and determining if the patient should undergo therapy with a compound of formula (I), or a pharmaceutically acceptable salt thereof, if a PI3Kδ mutation is present.

### BRIEF DESCRIPTION OF THE FIGURES

FIG. 1A shows percentage survival based on the defined mortality-endpoint (n=60) for Streptococcus Pneumoniae (S. Pneumoniae)-infected mice treated with the compound 6-(1H-indol-4-yl)-4-(5-{[4-(1-methylethyl)-1-piperazinyl]methyl}-1,3-oxazol-2-yl)-1H-indazole hydrochloride (white circles) and vehicle (black circles), analysed with Mantel-Cox test (**p<0.005) and median survival.

FIG. 1B is an Affymetrix GeneChip heatmap depicting genes that are significantly altered (minimum 1.5 fold-change; p<0.05) in lungs of mice treated with 6-(1H-indol-4-yl)-4-(5-{[4-(1-methylethyl)-1-piperazinyl]methyl}-1,3-oxazol-2-yl)-1H-indazole hydrochloride compared to vehicle controls at various timepoints after S. Pneumoniae-infection (n=6). Each band corresponds to a single probe and intensity signifies fold change, as indicated in the legend.

### DETAILED DESCRIPTION OF THE INVENTION

In one aspect, the present invention provides compounds of formula (I)

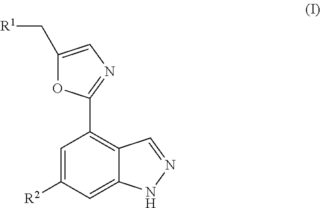

wherein

R¹ is

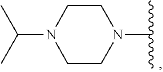 [SMILES: *N1CCN(C(C)C)CC1]

and

R₂ is

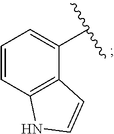 [SMILES: *c1cccc2[nH]ccc12]

or

R¹ is

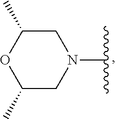 [SMILES: *N1C[C@@H](C)O[C@@H](C)C1]

and

R₂ is

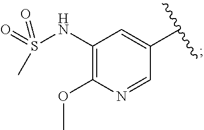 [SMILES: *c1cnc(OC)c(NS(C)(=O)=O)c1]

and pharmaceutically acceptable salts thereof for use in the treatment or prevention of respiratory infections, the treatment of airway damage, and/or the prevention of airway injury in patients with a PI3Kδ mutation.

In one embodiment, the present invention provides a compound which is 6-(1H-indol-4-yl)-4-(5-{[4-(1-methylethyl)-1-piperazinyl]methyl}-1,3-oxazol-2-yl)-1H-indazole:

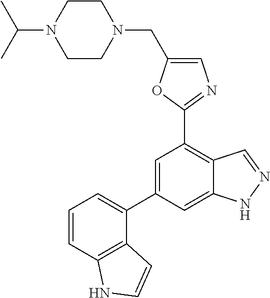 [SMILES: CC(C)N1CCN(Cc2cnc(-c3cc(-c4cccc5[nH]ccc45)cc4[nH]ncc34)o2)CC1]

or a pharmaceutically acceptable salt thereof for use in the treatment or prevention of a respiratory infection, the treatment of airway damage, and/or the prevention of airway injury in a patient with a PI3Kδ mutation.

In another embodiment, the present invention provides a compound which is 6-(1H-indol-4-yl)-4-(5-{[4-(1-methylethyl)-1-piperazinyl]methyl}-1,3-oxazol-2-yl)-1H-indazole hemi succinate for use in the treatment or prevention of a respiratory infection, the treatment of airway damage, and/or the prevention of airway injury in a patient with a PI3Kδ mutation.

In another embodiment, the present invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonannide:

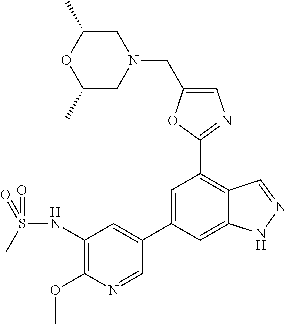 [SMILES: COc1ncc(-c2cc(-c3ncc(CN4C[C@@H](C)O[C@@H](C)C4)o3)c3cn[nH]c3c2)cc1NS(C)(=O)=O]

or a pharmaceutically acceptable salt thereof for use in the treatment or prevention of a respiratory infection, the treatment of airway damage, and/or the prevention of airway injury in a patient with a PI3Kδ mutation.

In another embodiment, the present invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient with a PI3Kδ mutation.

In another aspect, the present invention provides compounds of formula (I) as defined above and pharmaceutically acceptable salts thereof for use in the treatment or prevention of respiratory infections, the treatment of airway damage, and/or the prevention of airway injury in patients, comprising:
a) assaying samples from the patients,
b) determining if the patients have a PI3Kδ mutation, and
c) administering a therapeutically effective amount of a compound of formula (I) or a pharmaceutically acceptable salt thereof to the patients if they have a PI3Kδ mutation.

In one embodiment, the present invention provides a compound which is 6-(1H-indol-4-yl)-4-(5-{[4-(1-methylethyl)-1-piperazinyl]methyl}-1,3-oxazol-2-yl)-1H-indazole or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of 6-(1H-indol-4-yl)-4-(5-{[4-(1-methylethyl)-1-piperazinyl]methyl}-1,3-oxazol-2-yl)-1H-indazole or a pharmaceutically acceptable salt thereof to the patient if they have a PI3Kδ mutation.

In another embodiment, the present invention provides a compound which is 6-(1H-indol-4-yl)1)-4-(5-{[4-(1-methylethyl)-1-piperazinyl]methyl}-1,3-oxazol-2-yl)-1H-indazole hemi succinate for use in the treatment or prevention of a respiratory infection, the treatment of airway damage, and/or the prevention of airway injury in a patient, comprising:
a) assaying a sample from the patient,
b) determining if the patient has a PI3Kδ mutation, and administering a therapeutically effective amount of 6-(1H-indol-4-yl)-4-(5-{[4-(1-methylethyl)-1-piperazinyl]methyl}-1,3-oxazol-2-yl)-1H-indazole hemi succinate to the patient if they have a PI3Kδ mutation.

In another embodiment, the present invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of 6-(1H-indol-4-yl)-4-(5-{[4-(1-methylethyl)-1-piperazinyl]methyl}-1,3-oxazol-2-yl)-1H-indazole or a pharmaceutically acceptable salt thereof to the patient if they have a PI3Kδ mutation.

In a further embodiment, the present invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof to the patient if they have a PI3Kδ mutation.

In another aspect, the invention provides compounds of formula (I) as defined above and pharmaceutically acceptable salts thereof for use in the treatment or prevention of respiratory infections, the treatment of airway damage, and/or the prevention of airway injury in patients classified as responders, wherein a responder is characterised by the presence of a PI3Kδ mutation.

In one embodiment, the invention provides a compound which is 6-(1H-indol-4-yl)-4-(5-{[4-(1-methylethyl)-1-piperazinyl]methyl}-1,3-oxazol-2-yl)-1H-indazole or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides a compound which is 6-(1H-indol-4-yl)-4-(5-{[4-(1-methylethyl)-1-piperazinyl]methyl}-1,3-oxazol-2-yl)-1H-indazole hemi succinate for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient classified as a responder, wherein a responder is characterised by the presence of a PI3Kδ mutation.

As used herein, the term “responder” means someone who is identified (using a particular test or method) to be more likely to derive benefit in response to treatment (e.g. positive response to drug, reduction in adverse events, etc.). It is understood that not all people who have been identified as a responder will necessarily derive benefit, but as a patient class, they are more likely to do so. For example, it may be that out of the total untested diseased population, approximately 80% of that population derive benefit from a drug, but out of the group of “responders” (i.e. those individuals who have been tested, and identified as a responder according to the set criteria) approximately 99% will derive benefit.

In a further aspect, the invention provides a method of evaluating therapy with compounds of formula (I) as defined above and pharmaceutically acceptable salts thereof, comprising:
a) obtaining a sample from the patient,
b) testing for a PI3Kδ mutation, and
c) determining if the patient should undergo therapy with a compound of formula (I) as defined above, or a pharmaceutically acceptable salt thereof, if a PI3Kδ mutation is present.

In one embodiment, the invention provides a method of evaluating therapy with 6-(1H-indol-4-yl)-4-(5-{[4-(1-methylethyl)-1-piperazinyl]methyl}-1,3-oxazol-2-yl)-1H-indazole or a pharmaceutically acceptable salt thereof, comprising:
a) obtaining a sample from the patient,
b) testing for a PI3Kδ mutation, and
c) determining if the patient should undergo therapy with 6-(1H-indol-4-yl)-4-(5-{[4-(1-methylethyl)-1-piperazinyl]methyl}-1,3-oxazol-2-yl)-1H-indazole or a pharmaceutically acceptable salt thereof if a PI3Kδ mutation is present.

In another embodiment, the invention provides a method of evaluating therapy with 6-(1H-indol-4-yl)-4-(5-{[4-(1-methylethyl)-1-piperazinyl]methyl}-1,3-oxazol-2-yl)-1H-indazole hemi succinate, comprising:
a) obtaining a sample from the patient,
b) testing for a PI3Kδ mutation, and
c) determining if the patient should undergo therapy with 6-(1H-indol-4-yl)-4-(5-{[4-(1-methylethyl)-1-piperazinyl]methyl}-1,3-oxazol-2-yl)-1H-indazole hemi succinate if a PI3Kδ mutation is present.

In another embodiment, the invention provides a method of evaluating therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof, comprising:
a) obtaining a sample from the patient,
b) testing for a PI3Kδ mutation, and
c) determining if the patient should undergo therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof if a PI3Kδ mutation is present.

In a further embodiment, the invention provides a method of evaluating therapy with N-[5-[4-(5-{[(2R,65)-2,6-dimethyl-4-morpholinyl]methyl}-1,3-oxazol-2-yl)-1H-indazol-6-yl]-2-(methyloxy)-3-pyridinyl]methanesulfonamide, comprising:
a) obtaining a sample from the patient,
b) testing for a PI3Kδ mutation, and
c) determining if the patient should undergo therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide if a PI3Kδ mutation is present.

As used herein, the term “evaluating therapy” means determining whether therapy with a compound of formula (I), or a pharmaceutically acceptable salt thereof, would be beneficial to a patient.

Included within the scope of the invention is the use of all solvates (including hydrates), complexes, polymorphs, prodrugs and radiolabelled derivatives of a compound of formula (I) or a pharmaceutically acceptable salt thereof.

Compounds of formula (I) may be administered as a pharmaceutically acceptable salt. As used herein, the term “pharmaceutically acceptable salt” refers to a salt that retains the desired biological activity of the compound and exhibits minimal undesired toxicological effects. Pharmaceutically acceptable salts of compounds may be used to impart greater stability or solubility to a molecule thereby facilitating formulation into a dosage form. These pharmaceutically acceptable salts may be prepared in situ during the final isolation and purification of the compound, or by separately reacting the purified compound, or a non-pharmaceutically acceptable salt thereof, with a suitable base or acid. For a review on suitable salts see Berge et al., J. Pharm. Sci., 1977, 66, 1-19. In one embodiment, the invention provides the use of a pharmaceutically acceptable salt of 6-(1H-indol-4-yl)-4-(5-{[4-(1-methylethyl)-1-piperazinyl]methyl}-1,3-oxazol-2-yl)-1H-indazole. In another embodiment, the invention provides the use of 6-(1H-indol-4-yl)-4-(5-{[4-(1-methylethyl)-1-piperazinyl]methyl}-1,3-oxazol-2-yl)-1H-indazole hemi succinate. In another embodiment, the invention provides the use of a pharmaceutically acceptable salt of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide. In a further embodiment, the invention provides the use of N-[5-[4-(5-{[(2R,65)-2,6-dimethyl-4-morpholinyl]methyl}-1,3-oxazol-2-yl)-1H-indazol-6-yl]-2-(methyloxy)-3-pyridinyl]methanesulfonamide as the free base.

### Compound Preparation

The compounds and pharmaceutically acceptable salts for use according to the invention may be made by a variety of methods, including standard chemistry. For example, 6-(1H-indol-4-yl)-4-(5-{[4-(1-methylethyl)-1-piperazinyl]methyl}-1,3-oxazol-2-yl)-1H-indazole, N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide, and their pharmaceutically acceptable salts may be prepared as described in WO2010/125082, WO2012/055846 and/or WO2012/032067.

### Methods of Use

The methods of treatment of the present invention comprise administering a safe and effective amount of a compound of formula (I) or a pharmaceutically acceptable salt thereof to a patient in need thereof.

The present invention provides the treatment or prevention of a respiratory infection, the treatment of airway damage, and/or the prevention of airway injury in a patient with a PI3Kδ mutation. In one embodiment, the present invention provides the treatment or prevention of a respiratory infection. In another embodiment, the present invention provides the treatment of airway damage. In a further embodiment, the present invention provides the prevention of airway injury.

Patients with a PI3Kδ mutation may be identified by methods known to those skilled in the art, for example, methods involving polymerase chain reaction (PCR).

In one embodiment, the present invention provides a compound of formula (I) or a pharmaceutically acceptable salt thereof for use in the treatment of a respiratory infection. In a further embodiment, the present invention provides a compound of formula (I) or a pharmaceutically acceptable salt thereof for use in the prevention of a respiratory infection.

As used herein, “treat ” in reference to a disorder means: (1) to ameliorate the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As used herein, “safe and effective amount” in reference to a compound of formula (I) or a pharmaceutically acceptable salt thereof, or other pharmaceutically-active agent, means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

Patients with a PI3Kδ mutation may be particularly susceptible to developing respiratory infections and/or exacerbations of respiratory infections. Such respiratory infections may be the result of bacterial infections including, for example, infections by S. Pneumoniae, H. Influenzae, and/or M. Catarrhalis; viral infections including, for example, infections by influenza, rhinovirus, respiratory syncytial virus (RSV), human parainfluenza virus (HPIV), adenovirus and/or coronavirus; and other non-viral respiratory infections including aspergillosis and/or leishmaniasis. In one embodiment, the respiratory infection is a bacterial infection. In another embodiment, patients with a PI3Kδ mutation may be particularly susceptible to developing respiratory infections and/or exacerbations of respiratory infections as a result of bacterial infections by S. Pneumoniae, H. Influenzae, and/or M. Catarrhalis.

Bacterial respiratory infections which may be treated according to the invention include rhinitis, sinusitis, laryngitis, bronchitis, bronciolitis, tonsillitis, pneumonia and/or tuberculosis.

In one aspect, the invention is directed to the treatment of patients with a PI3Kδ mutation and a underlying disorder. Such patients may have an underlying disorder such as chronic obstructive pulmonary disease (COPD), asthma, bronchiectasis, cystic fibrosis or idiopathic fibrosis (IPF), or a compromised immune system. In one embodiment, the invention is directed to the treatment of bacterial respiratory infections patients with a PI3Kδ mutation and a underlying disorder. In another embodiment, the invention is directed to the treatment of patients with a PI3Kδ mutation and COPD. In a further embodiment, the invention is directed to the treatment of bacterial respiratory infections patients with a PI3Kδ mutation and COPD.

Patients with a PI3Kδ mutation may be particularly susceptible to an exacerbation of a respiratory infection. As used herein, the term “exacerbation of a respiratory infection” refers to a respiratory infection characterised by the worsening of an underlying persistent respiratory infection, including bacterial infections, viral infections and/or other non-viral respiratory infections. In one embodiment, the present invention provides a compound of formula (I) or a pharmaceutically acceptable salt thereof for use in the treatment or prevention of an exacerbation of a respiratory infection in a patient with a PI3Kδ mutation.

Patients with a PI3Kδ mutation may be particularly susceptible to developing airway damage and/or airway injury. As used herein, the term “airway damage” refers to damage to the airway wall, large and small airways, and/or lung parenchyma which is present at the time a patient commences treatment. Airway damage, such as inflammation, scarring and/or remodelling, may be caused by, for example, repeated respiratory infections in a patient with a PI3Kδ mutation. As used herein, the term “airway injury” refers to damage, or further damage, to the airway wall, large and small airways, and/or lung parenchyma which may develop in a patient if treatment does not occur.

In one embodiment, the respiratory infection is a sinopulmonary infection.

As described herein, the invention provides the treatment or prevention of respiratory infections, the treatment of airway damage, and/or the prevention of airway injury in patients with a PI3Kδ mutation. In one embodiment, the patients with a PI3Kδ mutation may have one or more nucleotide exchanges as compared with wild type PI3Kδ. In another embodiment, the patients with a PI3Kδ mutation may have from one to three nucleotide exchanges as compared with wild type PI3Kδ. In another embodiment, the patients with a PI3Kδ mutation may have one or two nucleotide exchanges as compared with wild type PI3Kδ. In a further embodiment, the patients with a PI3Kδ mutation may have one nucleotide exchange as compared with wild type PI3Kδ.

In one embodiment, the patient with a PI3Kδ mutation is heterozygous. As used herein, the term “heterozygous” in reference to a PI3Kδ mutation means that the mutation occurs in only one of a pair of alleles.

In one embodiment, the PI3Kδ mutation is a germline mutation.

In one embodiment, the PI3Kδ mutation is a non-synonymous mutation. As used herein, “non-synonymous mutation” refers to a nucleotide mutation which results in a change in the amino acid sequence of the PI3Kδ protein as compared with the wild type PI3Kδ protein.

In one embodiment, the PI3Kδ mutation is a missense mutation. As used herein, “missense mutation” is a type of non-synonymous mutation in which a point mutation of a single nucleotide results in a codon which codes for a different amino acid in the sequence of the PI3Kδ protein as compared with the wild type PI3Kδ protein.

In one embodiment, the PI3Kδ mutation results in the substitution of one or more amino acids in the amino acid sequence of the PI3Kδ protein. In another embodiment, the PI3Kδ mutation results in the substitution of from one to three amino acids in the amino acid sequence of the PI3Kδ protein. In another embodiment, the PI3Kδ mutation results in the substitution of one or two amino acids in the amino acid sequence of the PI3Kδ protein. In a further embodiment, the PI3Kδ mutation results in the substitution of one amino acid in the amino acid sequence of the PI3Kδ protein.

Class 1A PI3K molecules comprise a p110 catalytic subunit and a regulatory subunit. In one embodiment, the PI3Kδ mutation results in the substitution of one or more amino acids in the amino acid sequence of the p1106 catalytic subunit. In another embodiment, the PI3Kδ mutation results in the substitution of one or more amino acids in the amino acid sequence of the regulatory subunit. In a further embodiment, the PI3Kδ mutation results in the substitution of one or more amino acids in the amino acid sequence of the p85α regulatory subunit.

In one embodiment, the PI3Kδ mutation results in the substitution of one or more amino acids in the amino acid sequence inside the catalytic functional domain of the PI3Kδ protein. In another embodiment, the PI3Kδ mutation results in the substitution of from one to three amino acids in the amino acid sequence inside the catalytic functional domain of the PI3Kδ protein. In another embodiment, the PI3Kδ mutation results in the substitution of one or two amino acids in the amino acid sequence inside the catalytic functional domain of the PI3Kδ protein. In a further embodiment, the PI3Kδ mutation results in the substitution of one amino acid in the amino acid sequence inside the catalytic functional domain of the PI3Kδ protein.

In one embodiment, the PI3Kδ mutation results in the substitution of one or more amino acids in the amino acid sequence outside the catalytic functional domain of the PI3Kδ protein. In another embodiment, the PI3Kδ mutation results in the substitution of from one to three amino acids in the amino acid sequence outside the catalytic functional domain of the PI3Kδ protein. In another embodiment, the PI3Kδ mutation results in the substitution of one or two amino acids in the amino acid sequence outside the catalytic functional domain of the PI3Kδ protein. In a further embodiment, the PI3Kδ mutation results in the substitution of one amino acid in the amino acid sequence outside the catalytic functional domain of the PI3Kδ protein.

In one embodiment, the PI3Kδ mutation results in the substitution of one or more amino acids in the amino acid sequence in the C2 domain of the PI3Kδ protein.

In one embodiment, the PI3Kδ mutation results in the substitution of one or more amino acids in the amino acid sequence in the helical domain of the PI3Kδ protein.

In one embodiment, the PI3Kδ mutation results in the substitution of one or more amino acids in the amino acid sequence in the C-lobe of the kinase domain of the PI3Kδ protein.

In one embodiment, the PI3Kδ mutation results in the substitution of glutamic acid for lysine. In another embodiment, the PI3Kδ mutation results in the substitution of glutamic acid for lysine at codon 1021 (E1021K).

In one embodiment, the PI3Kδ mutation results in a single base-pair missense mutation m.3256G>A in the mRNA (wherein the nucleotide number is based on the sequence data on GenBank: NM_005026).

In one embodiment, the PI3Kδ mutation is c.3061G>A.

In one embodiment, the PI3Kδ mutation results in the substitution of asparagine for lysine. In another embodiment, the PI3Kδ mutation results in the substitution of asparagine for lysine at codon 334 (N334K).

In one embodiment, the PI3Kδ mutation results in a C to A mutation at cDNA position 1002 (wherein the nucleotide number is based on the sequence data on GenBank: NM_005026).

In one embodiment, the PI3Kδ mutation results in the substitution of glutamic acid for lysine at codon 525 (E525K).

In one embodiment, the PI3Kδ mutation results in a G to A mutation at nucleotide 1573 (wherein the nucleotide number is based on the sequence data on GenBank: NM_005026).

In one embodiment, the PI3Kδ mutation results in a mutation of the PI3K catalytic subunit c. 1246T>C, p. C416R.

In one embodiment, the PI3Kδ mutation results in a PIK3R1 mRNA splice mutation resulting in the exclusion of exon 10 and thus the deletion of amino acid residues 434-475 of the p85a regulatory subunit.

Mutations in PI3Kδ may lead to inappropriate PI3Kδ activity. Specifically, PI3Kδ mutations may lead to an increase in PI3Kδ activity as compared to wild type PI3Kδ protein (an activating mutation) or a decrease in PI3Kδ activity as compared to wild type PI3Kδ protein (a de-activating mutation). In one embodiment, the PI3Kδ mutation is an activating mutation. In a further embodiment, the PI3Kδ mutation is a de-activating mutation.

The compound or a pharmaceutically acceptable salt thereof may be administered by any suitable route of administration, in particular inhaled administration.

The compound or a pharmaceutically acceptable salt thereof may be administered according to a dosing regimen wherein a number of doses are administered at varying intervals of time for a given period of time. For example, doses may be administered one, two, three, or four times per day. In one embodiment, a dose is administered twice per day (BID).

Doses may be administered until the desired therapeutic effect is achieved or indefinitely to maintain the desired therapeutic effect. Suitable dosing regimens, including the duration such regimens are administered, may depend on the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages for oral administration may range from about 0.1 mg to about 20 mg, for example from about 0.1 mg to about 10 mg such as about 0.4 mg to about 7 mg. For example, a dose of from about 0.1 mg to about 5 mg, for example from about 0.2 mg to about 3.5 mg such as from about 0.25 mg to about 3 mg, may be administered BID per patient.

In one aspect, the invention provides a compound of formula (I) or a pharmaceutically acceptable salt thereof for use in the treatment or prevention of a respiratory infection, the treatment of airway damage, and/or the prevention of airway injury in a patient with a PI3Kδ mutation.

In one embodiment, the invention provides the use of a compound of formula (I) or a pharmaceutically acceptable salt thereof in the manufacture of a medicament for use in the treatment or prevention of a respiratory infection, the treatment of airway damage, and/or the prevention of airway injury in a patient with a PI3Kδ mutation.

In another embodiment, the invention provides a method of treating or preventing a respiratory infection, treating airway damage, and/or preventing airway injury in a patient with a PI3Kδ mutation comprising administering a safe and effective amount of a compound of formula (I) or a pharmaceutically acceptable salt thereof to a patient in need thereof.

In another embodiment, the present invention provides a compound of formula (I) or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of a compound of formula (I) or a pharmaceutically acceptable salt thereof to the patient if they have a PI3Kδ mutation.

In another embodiment, the invention provides a compound of formula (I) or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides use of a compound of formula (I) or a pharmaceutically acceptable salt thereof in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a method of evaluating therapy with a compound of formula (I) or a pharmaceutically acceptable salt thereof, comprising:
a) obtaining a sample from the patient,
b) testing for a PI3Kδ mutation, and
c) determining if the patient should undergo therapy with a compound of formula (I) or a pharmaceutically acceptable salt thereof if a PI3Kδ mutation is present.

In another aspect, the invention provides a compound which is 6-(1H-indol-4-yl)-4-(5-{[4-(1-methylethyl)-1-piperazinyl]methyl}-1,3-oxazol-2-yl)-1H-indazole or a pharmaceutically acceptable salt thereof for use in the treatment or prevention of a respiratory infection, the treatment of airway damage, and/or the prevention of airway injury in a patient with a PI3Kδ mutation.

In one embodiment, the invention provides the use of a compound which is 6-(1H-indol-4-yl)-4-(5-{[4-(1-methylethyl)-1-piperazinyl]methyl}-1,3-oxazol-2-yl)-1H-indazole or a pharmaceutically acceptable salt thereof in the manufacture of a medicament for use in the treatment or prevention of a respiratory infection, the treatment of airway damage, and/or the prevention of airway injury in a patient with a PI3Kδ mutation.

In another embodiment, the invention provides a method of treating or preventing a respiratory infection, treating airway damage, and/or preventing airway injury in a patient with a PI3Kδ mutation comprising administering a safe and effective amount of 6-(1H-indol-4-yl)-4-(5-{[4-(1-methylethyl)-1-piperazinyl]methyl}-1,3-oxazol-2-yl)-1H-indazole or a pharmaceutically acceptable salt thereof to a patient in need thereof.

In another embodiment, the present invention provides 6-(1H-indol-4-yl)-4-(5-{[4-(1-methylethyl)-1-piperazinyl]methyl}-1,3-oxazol-2-yl)-1H-indazole or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of 6-(1H-indol-4-yl)-4-(5-{[4-(1-methylethyl)-1-piperazinyl]methyl}-1,3-oxazol-2-yl)-1H-indazole or a pharmaceutically acceptable salt thereof to the patient if they have a PI3Kδ mutation.

In another embodiment, the invention provides a 6-(1H-indol-4-yl)-4-(5-{[4-(1-methylethyl)-1-piperazinyl]methyl}-1,3-oxazol-2-yl)-1H-indazole or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides use of 6-(1H-indol-4-yl)-4-(5-{[4-(1-methylethyl)-1-piperazinyl]methyl}-1,3-oxazol-2-yl)-1H-indazole or a pharmaceutically acceptable salt thereof in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a method of evaluating therapy with 6-(1H-indol-4-yl)-4-(5-{[4-(1-methylethyl)-1-piperazinyl]methyl}-1,3-oxazol-2-yl)-1H-indazole or a pharmaceutically acceptable salt thereof, comprising:
a) obtaining a sample from the patient,
b) testing for a PI3Kδ mutation, and
c) determining if the patient should undergo therapy with 6-(1H-indol-4-yl)-4-(5-{[4-(1-methylethyl)-1-piperazinyl]methyl}-1,3-oxazol-2-yl)-1H-indazole or a pharmaceutically acceptable salt thereof if a PI3Kδ mutation is present.

In another aspect, the invention provides a compound which is 6-(1H-indol-4-yl)-4-(5-{[4-(1-methylethyl)-1-piperazinyl]methyl}-1,3-oxazol-2-yl)-1H-indazole hemi succinate thereof for use in the treatment or prevention of a respiratory infection, the treatment of airway damage, and/or the prevention of airway injury in a patient with a PI3Kδ mutation.

In one embodiment, the invention provides the use of a compound which is 6-(1H-indol-4-yl)-4-(5-{[4-(1-methylethyl)-1-piperazinyl]methyl}-1,3-oxazol-2-yl)-1H-indazole hemi succinate in the manufacture of a medicament for use in the treatment or prevention of a respiratory infection, the treatment of airway damage, and/or the prevention of airway injury in a patient with a PI3Kδ mutation.

In another embodiment, the invention provides a method of treating or preventing a respiratory infection, treating airway damage, and/or preventing airway injury in a patient with a PI3Kδ mutation comprising administering a safe and effective amount of 6-(1H-indol-4-yl)-4-(5-{[4-(1-methylethyl)-1-piperazinyl]methyl}-1,3-oxazol-2-yl)-1H-indazole hemi succinate to a patient in need thereof.

In another embodiment, the present invention provides 6-(1H-indol-4-yl)-4-(5-{[4-(1-methylethyl)-1-piperazinyl]methyl}-1,3-oxazol-2-yl)-1H-indazole hemi succinate for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of 6-(1H-indol-4-yl)-4-(5-{[4-(1-methylethyl)-1-piperazinyl]methyl}-1,3-oxazol-2-yl)-1H-indazole hemi succinate to the patient if they have a PI3Kδ mutation.

In another embodiment, the invention provides a 6-(1H-indol-4-yl)-4-(5-{[4-(1-methylethyl)-1-piperazinyl]methyl}-1,3-oxazol-2-yl)-1H-indazole hemi succinate for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides use of 6-(1H-indol-4-yl)-4-(5-{[4-(1-methylethyl)-1-piperazinyl]methyl}-1,3-oxazol-2-yl)-1H-indazole hemi succinate in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a method of evaluating therapy with 6-(1H-indol-4-yl)-4-(5-{[4-(1-methylethyl)-1-piperazinyl]methyl}-1,3-oxazol-2-yl)-1H-indazolehemi succinate, comprising:
a) obtaining a sample from the patient,
b) testing for a PI3Kδ mutation, and
c) determining if the patient should undergo therapy with 6-(1H-indol-4-yl)-4-(5-{[4-(1-methylethyl)-1-piperazinyl]methyl}-1,3-oxazol-2-yl)-1H-indazole hemi succinate if a PI3Kδ mutation is present.

In another aspect, the invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof for use in the treatment or prevention of a respiratory infection, the treatment of airway damage, and/or the prevention of airway injury in a patient with a PI3Kδ mutation.

In one embodiment, the invention provides the use of a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof in the manufacture of a medicament for use in the treatment or prevention of a respiratory infection, the treatment of airway damage, and/or the prevention of airway injury in a patient with a PI3Kδ mutation.

In another embodiment, the invention provides a method of treating a respiratory infection, treating airway damage, and/or preventing airway injury in a patient with a PI3Kδ mutation comprising administering a safe and effective amount of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof to a patient in need thereof.

In another embodiment, the present invention provides N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of N-[5-[4-(5-{[(2R,65)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof to the patient if they have a PI3Kδ mutation.

In another embodiment, the invention provides N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides use of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a method of evaluating therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof, comprising:
a) obtaining a sample from the patient,
b) testing for a PI3Kδ mutation, and
c) determining if the patient should undergo therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide or a pharmaceutically acceptable salt thereof if a PI3Kδ mutation is present.

In a further aspect, the invention provides a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient with a PI3Kδ mutation.

In one embodiment, the invention provides the use of a compound which is N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide in the manufacture of a medicament for use in the treatment or prevention of a respiratory infection, the treatment of airway damage, and/or the prevention of airway injury in a patient with a PI3Kδ mutation.

In another embodiment, the invention provides a method of treating a respiratory infection, treating airway damage, and/or preventing airway injury in a patient with a PI3Kδ mutation comprising administering a safe and effective amount of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide to a patient in need thereof.

In another embodiment, the present invention provides N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient, comprising:
a) assaying a sample from the patient,
b) determining if the patient has a PI3Kδ mutation, and
c) administering a therapeutically effective amount of N-[5-[4-(5-{[(2R,65)-2,6-dimethyl-4-morpholinyl]methyl}-1,3-oxazol-2-yl)-1H-indazol-6-yl]-2-(methyloxy)-3-pyridinyl]methanesulfonamide to the patient if they have a PI3Kδ mutation.

In another embodiment, the invention provides N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide for use in the treatment or prevention of a respiratory infection, the treatment of airway damage, and/or the prevention of airway injury in a patient classified as a responder, wherein a responder is characterised by the presence of a PI3Kδ mutation.

In another embodiment, the invention provides use of N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide in the manufacture of a medicament for use in the treatment or prevention of a respiratory infection, the treatment of airway damage, and/or the prevention of airway injury in a patient classified as a responder, wherein a responder is characterised by the presence of a PI3Kδ mutation.

In a further embodiment, the invention provides a method of evaluating therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide, comprising:
a) obtaining a sample from the patient,
b) testing for a PI3Kδ mutation, and
c) determining if the patient should undergo therapy with N-[5-[4-(5-{[(2R,6S)-2,6-dimethyl-4-morpholinyl]methyl}-1,3-oxazol-2-yl)-1H-indazol-6-yl]-2-(methyloxy)-3-pyridinyl]methanesulfonamide if a PI3Kδ mutation is present.

### Compositions

The compounds of formula (I) and pharmaceutically acceptable salts thereof will normally, but not necessarily, be formulated into pharmaceutical compositions prior to administration to a patient. Accordingly, in another aspect the invention is directed to pharmaceutical compositions comprising a compound of formula (I) or a pharmaceutically acceptable salt thereof and one or more pharmaceutically-acceptable excipients for use in the treatment or prevention of respiratory infections, the treatment of airway damage, and/or the prevention of airway injury in patients with a PI3Kδ mutation.

The pharmaceutical compositions for use according to the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions for use according to the invention may be prepared and packaged in unit dosage form wherein each physically discrete unit contains a compound of formula (I) or a pharmaceutically acceptable salt thereof. When prepared in unit dosage form, the pharmaceutical compositions for use according to the invention typically may contain, for example, from 0.5 mg to 1 g, or from 1 mg to 700 mg, or from 5 mg to 100 mg of a compound of formula (I) or a pharmaceutically acceptable salt thereof.

The pharmaceutical compositions of for use according to the invention typically contain one compound of formula (I) or a pharmaceutically acceptable salt thereof.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of formula (I) or a pharmaceutically acceptable salt thereof when administered to a patient and interactions which would result in pharmaceutical compositions that are not pharmaceutically acceptable are avoided. In addition, each excipient must of course be pharmaceutically-acceptable e.g. of sufficiently high purity.

The compound of formula (I) or a pharmaceutically acceptable salt thereof and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of formula (I) or pharmaceutically acceptable salts thereof once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions for use according to the invention are prepared using techniques and methods known to those skilled in the art. Some of the methods commonly used in the art are described in Remington's Pharmaceutical Sciences (Mack Publishing Company).

Accordingly, in another aspect the invention is directed to a process for the preparation of a pharmaceutical composition for use according to comprising a compound of formula (I) or a pharmaceutically acceptable salt thereof and one or more pharmaceutically acceptable excipients which comprises mixing the ingredients. A pharmaceutical composition comprising a compound of formula (I) or a pharmaceutically acceptable salt thereof may be prepared by, for example, admixture at ambient temperature and atmospheric pressure.

In one embodiment, the compounds of formula (I) or pharmaceutically acceptable salts thereof will be formulated for oral administration. In another embodiment, the compounds of formula (I) or pharmaceutically acceptable salts thereof will be formulated for inhaled administration. In a further embodiment, the compounds of formula (I) or pharmaceutically acceptable salts thereof will be formulated for intranasal administration.

In one aspect, the invention is directed to the use of a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of formula (I) or pharmaceutically acceptable salts thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of formula (I) or pharmaceutically acceptable salts thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to the use of a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving the compound of formula (I) or a pharmaceutically acceptable salt thereof in a suitably flavoured aqueous solution, while elixirs are prepared through the use of a non-toxic alcoholic vehicle. Suspensions can be formulated by dispersing the compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to the use of a dosage form adapted for administration to a patient by inhalation. For example, as a dry powder, an aerosol, a suspension, or a solution composition.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (e.g. micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention the use of a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention there is provided the use of a pharmaceutical aerosol formulation wherein the propellant is selected from 1,1,1,2-tetrafluoroethane, 1,1,1,2,3,3,3-heptafluoro-n-propane and mixtures thereof.

The formulations for use according to the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or pharmaceutically acceptable salt thereof. Alternatively, the compound of formula (I) or pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations for use according to the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations for use according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

In a further aspect, the invention is directed to the use of a dosage form adapted for intranasal administration.

Formulations for administration to the nose may include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of formula (I) or pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Pharmaceutical compositions adapted for intranasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of formula (I) or a pharmaceutically acceptable salt thereof.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the compound of formula (I) or a pharmaceutically acceptable salt thereof may be employed with either a paraffinic or a water-miscible ointment base. Alternatively, the compound of formula (I) or pharmaceutically acceptable salt thereof may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

According to the invention, a compound of formula (I) or a pharmaceutically acceptable salt thereof may be used in combination with one or more other therapeutic agents, in the treatment or prevention of a respiratory infection, the treatment of airway damage, and/or the prevention of airway injury in a patient with a PI3Kδ mutation.

Suitable therapeutic agents for use in combination with a compound of formula (I) or a pharmaceutically acceptable salt thereof include one or more other therapeutic agents, for example selected from anti-inflammatory agents, anticholinergic agents (particularly an M₁/M₂/M₃ receptor antagonist), β₂-adrenoreceptor agonists, leukotriene antagonists, antiinfective agents, such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent, such as a corticosteroid or an NSAID, an anticholinergic agent, a β₂-adrenoreceptor agonist, a leukotriene antagonist, an anti-infective agent, such as an antibiotic or an antiviral, or an antihistamine for use in the treatment or prevention of a respiratory infection, the treatment of airway damage, and/or the prevention of airway injury in a patient with a PI3Kδ mutation. One embodiment of the invention encompasses combinations comprising a compound of formula (I) or a pharmaceutically acceptable salt thereof together with a β₂-adrenoreceptor agonist, and/or a leukotriene antagonist, and/or an anticholinergic, and/or a PDE-4 inhibitor, and/or an antihistamine, and/or DP2 antagonists, and/or a p38-kinase inhibitors and/or a DMARD (disease-modifying anti-rheumatic drug) for example, methotrexate, for use in the treatment or prevention of a respiratory infection, the treatment of airway damage, and/or the prevention of airway injury in a patient with a PI3Kδ mutation.

In one embodiment, the invention encompasses a method of treating or preventing a respiratory infection, treating airway damage, and/or preventing airway injury in a patient with a PI3Kδ mutation comprising administering a safe and effective amount of a combination comprising a compound of formula (I) or a pharmaceutically acceptable salt thereof together with one or more therapeutically active agents.

Certain compounds for use according to the invention may show selectivity for PI3Kδ over other PI3-kinases. The invention thus provides, in a further aspect, a combination comprising a compound of formula (I) or a pharmaceutically acceptable salt thereof which is selective for PI3Kδ together with a compound or pharmaceutically acceptable salt thereof which is selective for another PI3-kinase, for example PI3K_{Y}, for use in the treatment or prevention of a respiratory infection, the treatment of airway damage, and/or the prevention of airway injury in a patient with a PI3Kδ mutation.

One embodiment of the invention encompasses the use of combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of formula (I) or a pharmaceutically acceptable salt thereof together with a β₂-adrenoreceptor agonist for use in the treatment or prevention of a respiratory infection, the treatment of airway damage, and/or the prevention of airway injury in a patient with a PI3Kδ mutation.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single duastereomer such as the R,R-diastereomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment, long-acting β₂-adrenoreceptor agonists, for example, compounds which provide effective bronchodilation for about 12 hrs or longer, are preferred.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂-adrenoreceptor agonists include: 3-(4-{[6-({(2R)-2-hydroxy-2-[4-hydroxy-3-(hydroxymethyl)phenyl]ethyl}amino)hexyl]oxy}butyl)benzenesulfonamide;

3-(3-{[7-({(2R)-2-hydroxy-2-[4-hydroxy-3-hydroxymethyl)phenyl]ethyl}amino)heptyl]oxy}propyl)benzenesulfonamide;

4-{(1R)-2-[(6-{2-[(2,6-dichlorobenzyl)oxy]ethoxy}hexyl)amino]-1-hydroxyethyl}-2-(hydroxymethyl)phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]-ethyl]amino]ethyl]phenyl]formamide;

N-2{2-[4-(3-phenyl-4-methoxyphenyl)aminophenyl]ethyl}-2-hydroxy-2-(8-hydroxy-2(1H)-quinolinon-5-yl)ethylamine; and

5-[(R)-2-(2-{4-[4-(2-amino-2-methyl-propoxy)-phenylamino]-phenyl}-ethylamino)-1-hydroxy-ethyl]-8-hydroxy-1H -quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

In one embodiment, the invention encompasses a combination comprising a compound of formula (I) or a pharmaceutically acceptable salt thereof together with a leukotriene antagonist for use in the treatment or prevention of a respiratory infection, the treatment of airway damage, and/or the prevention of airway injury in a patient with a PI3Kδ mutation. Suitable leukotriene antagonists include, for example, montelukast.

Suitable anti-inflammatory agents include corticosteroids. Suitable corticosteroids which may be used in combination with the compounds of formula (I) or pharmaceutically acceptable salts thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-and rosta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethylcyclopropylcarbonyl)oxy-androsta-1,4-diene-17β-carbothioic acid S-cyanomethyl ester and 6α,9α-difluoro-11β-hyd roxy-16α-methyl-17α-(1-methylcyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α, 9α-difl uoro-11β-hydroxy-16α-methyl-17α-[(4-methyl-1,3-thiazole-5-carbonypont]-3-oxo-and rosta-1,4-diene-17p-carbothioic acid S-fluoromethyl ester, 6β,9α-difluoro-17α-[(2-furanylcarbonyl)oxy]-11β-hyd roxy-16α-methyl-3-oxo-and rosta-1,4-diene-17β-carbothioic acid S-fluoromethyl ester, 6α,9α-difluoro-11β-hyd roxy-16α-methyl-3-oxo-17α-(2,2,3,3-tetramethycyclopropylcarbonyl)oxy-and rosta-1,4-diene-17β-carbothioic acid S-cyanomethyl ester and 6α,9α-difluoro-11β-hydroxy-16α-methyl-17α-(1-methycyclopropylcarbonyl)oxy-3-oxo-and rosta-1,4-diene-17β-carbothioic acid S-fluoronmethyl ester.

In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid 5-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277. Further non-steroidal compounds are covered in: WO2006/000401, WO2006/000398 and WO2006/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), tryptase and elastase inhibitors, beta-2 integrin antagonists and adenosine receptor agonists or antagonists (e.g. adenosine 2a agonists), cytokine antagonists, or inhibitors of cytokine synthesis, or 5-lipoxygenase inhibitors.

In one embodiment, the invention provides the use of the compounds of formula (I) in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxphenyl)cyclohexa n-1-carboxyl is acid, 2-carbomethoxy-4-cyano-4-(3-cyclopropylmethoxy-4-difluoromethoxy-phenyl)cyclohexan-1-one and cis-[4-cyano-4-(3-cyclopropyl methoxy-4-difluoromethoxy-phenyl)cyclohexan-1-01]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd) (e.g. Example 399 or 544 disclosed therein). Further compounds are also disclosed in WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:

(3-endo)-3-(2,2-di-2-thienylethenyl)-8,8-dimethyl-8-azoniabicyclo[3.2.1]octane iodide;

(3-endo)-3-(2-cyano-2,2-diphenylethyl)-8,8-dimethyl-8-azoniabicyclo[3.2.1]octane bromide;

4-[hydroxy(diphenyl)methyl]-1-{2-[(phenylmethypoxy]ethyl}1-1-azoniabicyclo[2. 2. 2]octane bromide; and

(1R,5S)-3-(2-cyano-2,2-diphenylethyl)-8-methyl-8-{2-[(phenylmethyl}oxy]ethyl1-8-azoniabicyclo[3.2.1]octane bromide.

Other anticholinergic agents include compounds which are disclosed in U.S. patent application Ser. No. 60/487981 including, for example:

(3-endo)-3-(2,2-di-2-thienylethenyl)-8,8-dimethyl-8-azoniabicyclo[3.2.1]octane bromide;

(3-endo)-3-(2,2-diphenylethenyl)-8,8-dimethyl-8-azoniabicyclo[3.2.1]octane bromide;

(3-endo)-3-(2,2-diphenylethenyl)-8,8-dimethyl-8-azoniabicyclo[3.2.1]octane 4-methyl-benzenesulfonate;

(3-endo)-8,8-dimethyl-3-[2-phenyl-2-(2-thienyl)ethenyl]-8-azoniabicyclo[3.2.1]octane bromide; and/or

(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application Ser. No. 60/511009 including, for example:

(endo)-3-(2-methoxy-2,2-di-thiophen-2-yl-ethyl)-8,8-dimethyl-8-azonia-bicyclo[3.2.1]octane iodide;

3-((endo)-8-methyl-8-aza-bicyclo[3.2.1]oct-3-yl)-2,2-diphenyl-propionitrile;

(endo)-8-methyl-3-(2,2,2-triphenyl-ethyl)-8-aza-bicyclo[3.2.1]octane;

3-((endo)-8-methyl-8-aza-bicyclo[3.2.1]oct-3-yl)-2,2-diphenyl-propionamide;

3-((endo)-8-methyl-8-aza-bicyclo[3.2.1]oct-3-yl)-2,2-diphenyl-propionic acid;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

3-((endo)-8-methyl-8-aza-bicyclo[3.2.1]oct-3-yl)-2,2-diphenyl-propan-1-ol;

N-benzyl-3-((endo)-8-methyl-8-aza-bicyclo[3.2.1]oct-3-yl)-2,2-diphenyl-propionamide;

(endo)-3-(2-carbamoyl-2,2-diphenyl-ethyl)-8,8-dimethyl-8-azonia-bicyclo[3.2.1]octane iodide;

1-benzyl-3-[3-((endo)-8-methyl-8-aza-bicyclo[3.2.1]oct-3-yl)-2,2-diphenyl-propyl]-urea;

1-ethyl-3-[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-acetamide;

N-[3-((endo)-8-methyl-8-aza-bicyclo[3.2.1]oct-3-yl)-2,2-diphenyl-propyl]-benzamide;

3-((endo)-8-methyl-8-aza-bicyclo[3.2.1]oct-3-yl)-2,2-di-thiophen-2-yl-propionitrile;

(endo)-3-(2-cyano-2,2-di-thiophen-2-yl-ethyl)-8,8-dimethyl-8-azonia-bicyclo[3.2.1]octane iodide;

N-[3-((endo)-8-methyl-8-aza-bicyclo[3.2.1]oct-3-yl)-2,2-diphenyl-propyl]-benzene-sulfonamide;

[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-methane-sulfonamide; and/or

(endo)-3-{2,2-diphenyl-3-[(1-phenyl-methanoyl)-amino]-propyl}-8,8-dimethyl-8-azonia-bicyclo[3.2.1]octane bromide.

Further compounds include:

(endo)-3-(2-methoxy-2,2-di-thiophen-2-yl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

(endo)-3-(2-carbamoyl-2,2-diphenyl-ethyl)-8,8-dimethyl-8-azonia-bicyclo[3.2.1]octane iodide;

(endo)-3-(2-cyano-2,2-di-thiophen-2-yl-ethyl)-8,8-dimethyl-8-azonia-bicyclo[3.2.1]octane iodide; and/or

(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof together with an H1 antagonist for use in the treatment or prevention of a respiratory infection, the treatment of airway damage, and/or the prevention of airway injury in a patient with a PI3Kδ mutation. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

In one embodiment the invention provides a combination comprising a compound of formula (I) or a pharmaceutically acceptable salt thereof together with an anti-infective agent for use in the treatment or prevention of a respiratory infection, the treatment of airway damage, and/or the prevention of airway injury in a patient with a PI3Kδ mutation. The anti-infective agent may be an antibiotic, an antiviral or an antifungal. Examples of suitable antibiotics may include amoxicillin/clavulanate, flucloxacillin, cefalexin, cefixime, erythromycin, ciprofloxacin and tobramycin. Examples of suitable antivirals may include oseltamivir, zanamivir and ribavirin. Examples of suitable antifungals may include fluconazole and itraconazole.

In one embodiment the combination comprising a compound of formula (I) or a pharmaceutically acceptable salt thereof together with an anti-infective agent for use in the treatment or prevention of a respiratory infection, the treatment of airway damage, and/or the prevention of airway injury in a patient with a PI3Kδ mutation may be administered by inhalation. Examples of anti-infective agents particularly suitable for inhalation include those that may be inhaled or nebulized, for example, antibiotics such as tobramycin or ciprofloxacin, and antivirals such as zanamivir or ribavirin.

In one embodiment the invention provides a combination comprising a compound of formula (I) or a pharmaceutically acceptable salt thereof together with an anti-infective agent that has a compatible duration of action with the compound of formula (I). By the term “compatible duration of action” as used herein, is meant that the duration of action is such that both compounds may be administered to treat a particular patient, for example, they may be administered the same number of times each day such as once daily or 2, 3, 4 or 8 times.

The invention thus provides, in a further aspect, a combination comprising a compound of formula (I) or a pharmaceutically acceptable salt thereof together with a PDE4 inhibitor for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with a β₂-adrenoreceptor agonist for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with a leukotriene antagonist for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with a corticosteroid for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with a non-steroidal GR agonist for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with an anticholinergic for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with an antihistamine for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with a PDE4 inhibitor and a β₂-adrenoreceptor agonist for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with an anticholinergic and a PDE-4 inhibitor for use in the treatment or prevention of a respiratory infection, the treatment of airway damage, and/or the prevention of airway injury in a patient with a PI3Kδ mutation.

The invention thus provides, in a further aspect, a combination comprising a compound of formula (I) or a pharmaceutically acceptable salt thereof together with an anti-infective agent for use in the treatment or prevention of a respiratory infection, the treatment of airway damage, and/or the prevention of airway injury in a patient with a PI3Kδ mutation.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof together with another therapeutically active agent for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a PDE4 inhibitor for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a β₂-adrenoreceptor agonist for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a leukotriene antagonist for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a corticosteroid for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a non-steroidal GR agonist for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with an anticholinergic for use in the treatment or prevention of a respiratory infection, the treatment of airway damage, and/or the prevention of airway injury in a patient with a PI3Kδ mutation

The invention thus provides, in a further aspect, a pharmaceutical composition comprising a combination of a compound of formula (I) or a pharmaceutically acceptable salt thereof together with an antihistamine for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with a PDE4 inhibitor and a β₂-adrenoreceptor agonist for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with an anticholinergic and a PDE4 inhibitor for use in the treatment or prevention of a respiratory infection, the treatment of airway damage, and/or the prevention of airway injury in a patient with a PI3Kδ mutation.

The invention thus provides, in a further aspect, a pharmaceutical composition comprising a combination of a compound of formula (I) or a pharmaceutically acceptable salt thereof together with an anti-infective agent for use in the treatment or prevention of a respiratory infection, the treatment of airway damage, and/or the prevention of airway injury in a patient with a PI3Kδ mutation.

### EXAMPLES

### Example 1

### 6-(1H-Indol-4-yl)-4-(5-{[4-(1-methylethyl)-1-piperazinyl]methyl}-1,3-oxazol-2-yl)-1H-indazole hydrochloride for Use in Treating Streptococcus pneumoniae

Germ-free C57BL/6 male and female mice aged 10-12 weeks were intranasally administered with 0.2%Tween-80/saline vehicle or 0.2 mg/kg micronized 6-(1H-indol-4-yl)-4-(5-{[4-(1-methylethyl)-1-piperazinyl]methyl}-1,3-oxazol-2-yl)-1H-indazole hydrochloride in the same vehicle. Compound dosing was carried out twice daily for eleven days under anaesthesia using 3% isoflurane for induction and 2% isoflurane for maintenance. At the start of day two, and one hour after administering 6-(1H-indol-4-yl)-4-(5-{[4-(1-methylethyl)-1-piperazinyl]methyl}-1,3-oxazol-2-yl)-1H-indazole hydrochloride or vehicle, mice were anaesthetized with isoflurane as above and infected intranasally with 1×10⁷ CFU of S. pneumoniae strain TIGR4. S. Pneumoniae was obtained and prepared as described previously (see, for example, Infect. Immun. December 2011; 79(12): 4965-4976) and given in an inoculum of 50 μl PBS per mouse. Mice were monitored three times daily and assessed using a defined mortality-endpoint, where mice displaying three or more of the limiting clinical signs stated in Home Office Project Licence PPL 70/7661 were culled.

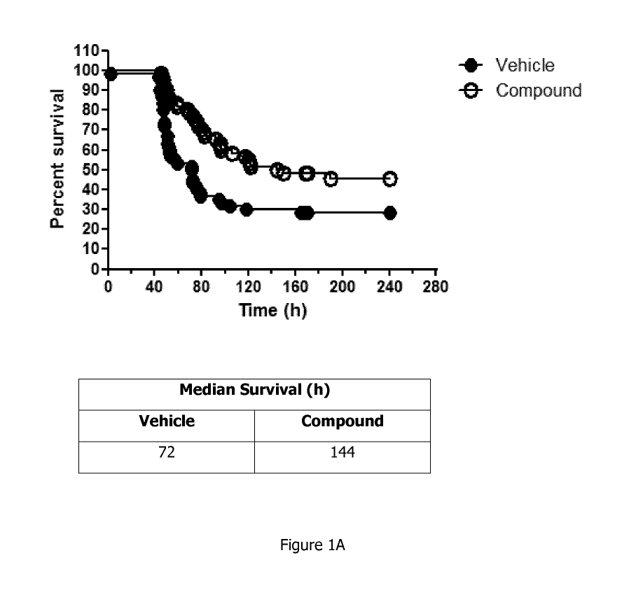

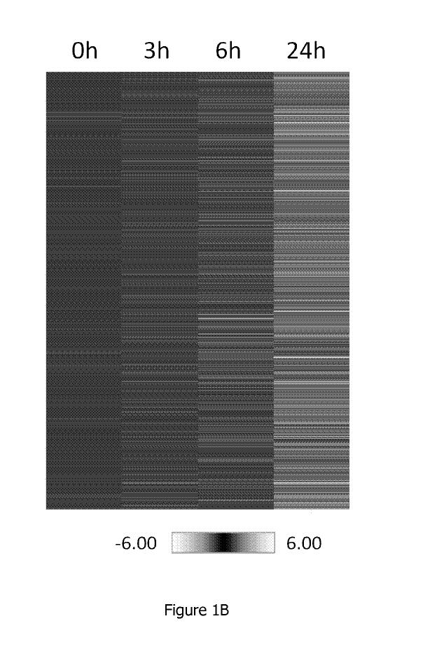

FIG. 1, comprising FIGS. 1A and 1B, demonstrates that in mice infected with Streptococcus pneumoniae, 6-(1H-indol-4-yl)-4-(5-{[4-(1-methylethyl)-1-piperazinyl]methyl}-1,3-oxazol-2-yl)-1H-indazole hydrochloride reduced a defined mortality-endpoint and altered lung gene expression compared to vehicle controls.

FIG. 1A shows percentage survival based on the defined mortality-endpoint (n=60) for Streptococcus Pneumoniae (S. Pneumoniae)-infected mice treated with the compound 6-(1H-indol-4-yl)-4-(5-{[4-(1-methylethyl)-1-piperazinyl]methyl}-1,3-oxazol-2-yl)-1H-indazole hydrochloride (white circles) and vehicle (black circles), analysed with Mantel-Cox test (**p<0.005) and median survival.

FIG. 1B is an Affymetrix GeneChip heatmap depicting genes that are significantly altered (minimum 1.5 fold-change; p<0.05) in lungs of mice treated with 6-(1H-indol-4-yl)-4-(5-{[4-(1-methylethyl)-1-piperazinyl]methyl}-1,3-oxazol-2-yl)-1H-indazole hydrochloride compared to vehicle controls at various timepoints after S. Pneumoniae-infection (n=6). Each band corresponds to a single probe and intensity signifies fold change, as indicated in the legend.

What is claimed is:

1-10. (canceled)

11. A method of treating or preventing a respiratory infection, treating airway damage, and/or preventing airway injury in a patient with a PI3Kδ mutation comprising administering a safe and effective amount of a compound of formula(I): 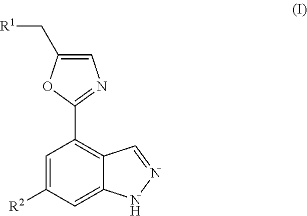
wherein
R₁ is 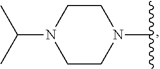 [SMILES: *N1CCN(C(C)C)CC1]
and
R² is 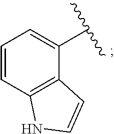 [SMILES: *c1cccc2[nH]ccc12]
or
R¹ is 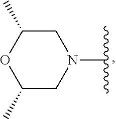 [SMILES: *N1C[C@@H](C)O[C@@H](C)C1]
and
R² is 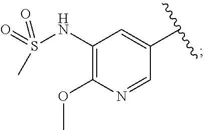 [SMILES: *c1cnc(OC)c(NS(C)(=O)=O)c1]
or a pharmaceutically acceptable salt thereof, to a patient in need thereof.

12-14. (canceled)

15. A method of evaluating therapy with a compound of formula (I): 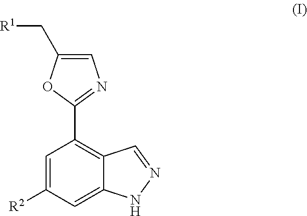
wherein
R¹ is 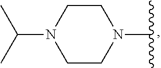 [SMILES: *N1CCN(C(C)C)CC1]
and
R² is 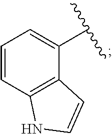 [SMILES: *c1cccc2[nH]ccc12]
or
R¹ is 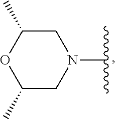 [SMILES: *N1C[C@@H](C)O[C@@H](C)C1]
and
R² is 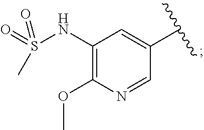 [SMILES: *c1cnc(OC)c(NS(C)(=O)=O)c1]
, or a pharmaceutically acceptable salt thereof, comprising:
a) obtaining a sample from the patient,
b) testing for a PI3Kδ mutation, and
c) determining if the patient should undergo therapy with a compound of formula (I), or a pharmaceutically acceptable salt thereof, if a PI3Kδ mutation is present.

16. A method according to claim 11, wherein the compound is 6-(1H-indol-4-yl)-4-(5-{[4-(1-methylethyl)-1-piperazinyl]methyl}-1,3-oxazol-2-yl)-1H-indazole, or a pharmaceutically acceptable salt thereof.

17. A method according to claim 11, wherein the compound is N-[544-(5-{[(2R,6S)-2,6-dimethyl-4-morpholinyl]methyl}-1,3-oxazol-2-yl)-1H-indazol-6-yl]-2-(methyloxy)-3-pyridinylynethanesulfonamide, or a pharmaceutically acceptable salt thereof.

18. A method according to claim 11, wherein the compound is (1H-indol-4-yl)-4-(5-{[4-(1-methylethyl)-1-piperazinyl]methyl}-1,3-oxazol-2-yl)-1H-indazole hemi succinate.

19. A method according to claim 11, wherein the compound is N-[544-(5-{[(2R,6S)-2,6-dimethyl-4-morpholinyl]methyl}-1,3-oxazol-2-yl)-1H-indazol-6-yl]-2-(methyloxy)-3-pyridinylynethanesulfonamide.

20. A method according to claim 11, wherein the treatment or prevention of a respiratory infection in a patient with a PI3Kδ mutation is the treatment or prevention of an exacerbation of respiratory infection.

21. A method according to claim 11, wherein the PI3Kδ mutation results in the substitution of one or more amino acids in the amino acid sequence of the PI3Kδ protein.

22. A method according to claim 11, wherein the PI3Kδ mutation results in the substitution of one or more amino acids in the amino acid sequence inside the catalytic functional domain of the PI3Kδ protein.

23. A method according to claim 11, wherein the PI3Kδ mutation is an activating mutation.
